(19) United States
(12) Patent Application Publication
(10) Pub. No.: US 2017/0239283 A1
(43) Pub. Date: Aug. 24, 2017

(54) USE OF STING AGONISTS TO TREAT VIRALLY-INDUCED AND PRE-MALIGNANT GROWTHS

(71) Applicant: Providence Health & Services - Oregon, Portland, OR (US)

(72) Inventors: Michael Gough, Portland, OR (US); Marka Crittenden, Portland, OR (US); Jason Baird, Portland, OR (US); Kristina Young, Portland, OR (US)

(73) Assignee: Providence Health & Services - Oregon, Portland, OR (US)

(21) Appl. No.: 15/439,707

(22) Filed: Feb. 22, 2017

Related U.S. Application Data
(60) Provisional application No. 62/298,789, filed on Feb. 23, 2016.

(51) Int. Cl. A61K 31/7076; A61K 31/7048; A61K 9/00

(52) U.S. Cl. CPC A61K 31/7076; A61K 9/0019; A61K 31/7048

(57) ABSTRACT

Disclosed herein are methods of treating or inhibiting a pre-malignant condition, a benign neoplasia, or a virally-induced growth in a subject. The methods include administering a composition including an effective amount of one or more Stimulator of Interferon Genes (STING) agonists, or a pharmaceutically acceptable salt or prodrug thereof, to the subject. In particular embodiments, the methods include administering one or more STING agonists that are a cyclic dinucleotide or a derivative thereof to the subject, for example cyclic diadenylate monophosphate, cyclic diguanylate monophosphate, or a derivative thereof.

### CROSS REFERENCE TO RELATED APPLICATION

This claims the benefit of U.S. Provisional Application No. 62/298,789, filed Feb. 23, 2016, which is incorporated herein by reference in its entirety.

### ACKNOWLEDGMENT OF GOVERNMENT SUPPORT

This invention was made with government support under Grant No. 1R01 CA182311-01A1 awarded by the National Institutes of Health. The government has certain rights in the invention.

### FIELD

This disclosure relates to compositions and methods for treating pre-malignant conditions and virally-induced growths.

### BACKGROUND

Most cancers are known to exhibit a pre-malignant state, where normal tissue organization is disrupted by an expanded population of mutated cells. This pre-malignant state can exist for years and may remain, resolve, or progress into cancer. Cancer screening programs, for example those based around the Papanicolaou (Pap) smear for cervical carcinoma, aim to identify these abnormal cells for intervention before further malignant transformation. Current therapies for a positive Pap smear include excision of the abnormal region, or ablation by freezing or lasers.

Immunotherapies, including interferon alpha and imiquimod have been added to excisional therapies to decrease the rate of recurrence; however, in randomized clinical trials it was found that neither approach impacts the rate of recurrence of cervical dysplasia (Pachman et al., Am. J. Obstet. Gynecol. 206:e41-47, 2012; Gostout et al., Int. J. Gynecol. Obstet. 74:207-210, 2001). Other pre-malignant growths, such as papillomas, veruccas, and condylomas, may not proceed to malignancy, but remain significant dermatological concerns.

### SUMMARY

Disclosed herein are methods of treating or inhibiting a pre-malignant condition or growth, a benign neoplasia, and/or a virally-induced growth in a subject. The methods include administering a composition including an effective amount of one or more Stimulator of Interferon Genes (STING) agonists, or a pharmaceutically acceptable salt or prodrug thereof, to the subject. In particular embodiments, the methods include administering to the subject a STING agonist that is a cyclic dinucleotide (CDN) or a derivative thereof, for example cyclic diadenylate monophosphate (CDA) or cyclic diguanylate monophosphate (CDG) or a derivative thereof.

In some examples, the subject has a pre-malignant condition such as cervical dysplasia, ductal carcinoma in situ, actinic keratosis, colon polyps, leukoplakia, erythroplakia, oral lichen planus, or Barrett's esophagus. In other examples, the subject has a virally-induced growth such as a papilloma, a condyloma, a verruca, or a wart. The virally-induced growth may be caused by infection with a human papillomavirus.

In some examples, the methods include administering the composition comprising the one or more STING agonists or a pharmaceutically acceptable salt or prodrug thereof to the subject intravenously, orally, or subcutaneously. In other examples, the composition is administered locally (at or near the site of the pre-malignant growth, benign neoplasia, or virally-induced growth), for example by local injection or topical application.

The foregoing and other features of the disclosure will become more apparent from the following detailed description, which proceeds with reference to the accompanying figures.

### BRIEF DESCRIPTION OF THE DRAWINGS

FIG. 1 is a digital image of a histological section of skin from a transgenic mouse model carrying the mutant tumor-driving genes Kras^{(G12D)} and p53 ^{(R172H)} that are controlled by expression of PDX-Cre. The section shows normal to dysplastic transformation, and is stained for STING and mouse immunoglobulins. Staining shows the immune reactivity in the dermis that underlies the cells of the epidermis.

FIGS. 2A and 2B are digital images of a mouse bearing two papillomas, of which one was treated with injection of cyclic diguanylate monophosphate (CDG) and the other left untreated (Control). FIG. 2A is a pair of images of the mouse before (left) and 18 hours after (right) treatment. FIG. 2B is a pair of images of histological sections of the untreated papilloma (left) and the treated papilloma 18 hours after treatment (right).

FIG. 3A is a series of representative digital images of mice bearing papillomas on days 0, 1, 4, 7, and 8 of treatment with CDG (CDN; first four rows) or imiquimod (IQ; last row). Mice were treated with injection on days 0, 1, 7, and 8.

FIG. 3B is a schematic showing treatment of Pdx-Cre^{+/-} Kras ^{(G12D)+/-} Trp53^{(R172H)+/-} mice with large papilloma. The mice were randomized to receive treatment with 25 μg CDG (CDN), 25 μg Imiquimod, or PBS vehicle, injected on days 0, 1, 7, and 8.

FIGS. 3C and 3D are graphs showing average size of papilloma through treatment. (FIG. 3C) and fold-change in papilloma size through treatment (FIG. 3D).

FIGS. 4A-C are a series of digital images showing infiltration of T cells following treatment with STING ligand. FIG. 4A shows images from Pdx-Cre^{+/-} Kras^{(G12D)+/-} Trp53^{(R172H)+/-} mice exhibiting papilloma injected with i) PBS vehicle, ii) 25 μg Imiquimod, or iii) 25 μg CDG and the site harvested 24 hours later for histology. Sections were stained for CD3 and DAPI nuclear counterstain. FIG. 4B shows images from a Pdx-Cre^{+/-} KRAS^{(G12D)+/-} Trp53^{(R172H)+/-} mouse exhibiting dual papilloma on opposite sides of the face injected with 25 μg CDG to one lesion and the other left untreated. Both papilloma sites were harvested 24 hours later and stained for CD3 and DAPI nuclear counterstain on the i) treated and ii) untreated opposite side papilloma. FIG. 4C shows CD3 and DAPI nuclear counterstain in a CDG-treated papilloma 14 days following initiation of treatment.

FIG. 5 is a digital image of a Western blot for iNOS, Arginase I, and GAPDH in murine bone marrow-derived macrophages treated with compounds targeting TLR4 (LPS), STING (CDA), NOD2 (PGNECndss), TLR7 (Imiquimod), TLR9 (class A CpG), or RIGI (ppp5′dsRNA), then treated with IFNγ (20 ng/ml) or IL-4 (20 ng/ml).

FIGS. 6A and B are a series of digital images showing STING expression in oral dysplasia. FIG. 6A shows STING expression in normal tonsil. Highlighted areas include positive iii) endothelia, iv) follicular dendritic cells, v) interdigitating cells, and vi) tonsilar crypt. FIG. 6B shows STING expression in tongue in i) benign dysplasia, ii) candida infection, iii) mild dysplasia/in situ, or iv) severe dysplasia.

FIGS. 7A-7E is a series of digital images showing STING expression in HPV-associated disease: normal uterus (FIG. 7A), benign dysplasia (FIG. 7B), condyloma (FIG. 7C), intraepithelial neoplasia grade 3 (AIN3, FIG. 7D), and cervical intraepithelial neoplasia grade 3 (CIN3, FIG. 7E).

FIG. 7F is a graph showing degree of STING staining scored on a scale from negative (−) to highly positive (+++). The graph shows a summary of the proportion of each histology with each staining pattern.

FIGS. 8A and 8B are a series of digital images showing STING expression in two examples of HPV⁺ head and neck squamous cell carcinoma (HNSCC) (FIG. 8A) and two examples of HPV⁺ HNSCC (FIG. 8B).

FIG. 8C is a graph showing intensity of STING expression in HPV⁺ and HPV⁻ HNSCC. Each symbol represents one patient. ****, p<0.0001

### DETAILED DESCRIPTION

I. Terms

Unless otherwise noted, technical terms are used according to conventional usage. Definitions of common terms in molecular biology may be found in Krebs et al., Lewin's Genes XI, published by Jones and Bartlett Learning, 2012 (ISBN 1449659853); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 2011 (ISBN 8126531789); and George P. Rédei, Encyclopedic Dictionary of Genetics, Genomics, and Proteomics, 2nd Edition, 2003 (ISBN: 0-471-26821-6).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of the invention, the following explanations of specific terms are provided:

Benign neoplasia: Tissue (such as a mass or growth) that does not invade surrounding tissue or metastasize, but in contrast to pre-malignant tissue, has limited potential for growth, and includes well-differentiated cells (e.g., cells with strong resemblance to normal cells in the tissue of origin).

Cyclic dinucleotide (CDN): CDNs are cyclic compounds including two nucleotides. Some CDNs are produced by bacteria and are signaling molecules involved in regulation of bacterial biofilm formation, motility, and virulence. They bind to STING protein and in some examples activate the interferon pathway. Exemplary CDNs include cyclic diadenylate monophosphate (CDA), cyclic diguanylate monophosphate (CDG), cyclic di-inosine monophosphate (CDI), and cyclic guanosine monophosphate-adenosine monophosphate (CGAMP). CDNs also include modified CDNs or derivatives of CDNs, such as CDNs that are less susceptible to phosphodiesterase degradation and/or having increased activity compared to naturally occurring STING ligands. Exemplary modified CDNs or CDN derivatives include those described in U.S. Pat. App. Publ. Nos. 2014/0341976 and 2014/0205653, both of which are incorporated herein by reference in their entirety.

Derivative: A compound that is derived from a base structure, for example, a compound that is derived from a similar compound or a compound that can arise from another compound, for example, if one atom is replaced with another atom or group of atoms. In some examples, the term “derivative” is also used for compounds that at least theoretically can be formed from a precursor compound. Derivatives are also referred to herein as “modified” compounds.

Papilloma: A neoplasm of epithelial origin having a morphology with a finger-like or cauliflower-like appearance. Papillomas are typically benign, but are pre-malignant in some cases. They are usually caused by infection with human papillomavirus (HPV) and commonly occur in the skin, genitals, mouth, eyes, and throat. Other types of papilloma, such as intraductal papilloma and choroid plexus papilloma are unknown cause.

Pharmaceutically acceptable carrier: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of one or more therapeutic compositions, such as one or more STING agonists, and/or additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of one or more non-toxic auxiliary substances, such as wetting or emulsifying agents, preservatives, pH buffering agents, and the like, for example sodium acetate or sorbitan monolaurate.

Pharmaceutically acceptable salt: A salt of a compound, which salts are derived from a variety of organic and inorganic counterions, for example, sodium, potassium, calcium, magnesium, ammonium, or tetraalkylammonium, or when the molecule contains a basic functionality, salts of organic or inorganic acids, such as hydrochloride, hydrobromide, tartrate, mesylate, acetate, maleate, or oxalate. Pharmaceutically acceptable acid addition salts are salts that retain the biological effectiveness of the free bases while formed by acid partners that are not biologically or otherwise undesirable, for example, inorganic acids (such as hydrochloric acid, hydrobromic acid, sulfuric acid, nitric acid, or phosphoric acid) or organic acids (such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, or salicylic acid). Pharmaceutically acceptable base addition salts include those derived from inorganic bases such as sodium, potassium, lithium, ammonium, calcium, magnesium, iron, zinc, copper, manganese, or aluminum salts. Exemplary salts are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethylaminoethanol, 2-diethylaminethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethypiperidine, polyamine resins, and the like. Exemplary organic bases are isopropylamine, diethylamine, ethanolamine, trimethylamine, dicyclohexylamine, choline, and caffeine. See, e.g., Handbook of Pharmaceutical Salts, Properties, Selection and Use, Wiley VCH (2002); Berge et al., J. Pharm. Sci. 66:1-19, 1977.

Pre-malignant: A condition of being potentially, but not yet, malignant. Pre-malignant tissue (such as a mass or growth) typically exhibits hyperplasia and/or dysplasia, for example, an increase or expansion of immature cells relative to normal tissue. Dysplasia frequently includes microscopic changes in tissue, such as anisocytosis (cells of unequal sizes), poikilocytosis (abnormally shaped cells), hyperchromatism, and/or an increase in cell division (for example, as identified by presence of mitotic figures). Pre-malignant growths do not have the ability to invade neighboring tissue or metastasize.

Prodrug: A compound that is transformed in vivo to yield the parent compound, for example by hydrolysis in the gut or enzymatic conversion in blood. Common examples include, but are not limited to ester and amide forms of a compound having an active form bearing a carboxylic acid moiety. See, e.g., Prodrugs as Novel Delivery Systems, Eds., Higuchi and Stella, ACS Symposium Series, Vol. 14, 1975; Bioreversible Carriers in Drug Design, ed. Roche, Pergamon Press, 1987.

STING (Stimulator of Interferon Genes): Also known as Transmembrane protein 173 or TMEM173. A protein with five transmembrane domains that is a regulator of innate immune response to viral and bacterial infection. STING is a receptor that binds cytosolic nucleic acids and activates type I interferon responses. STING nucleic acid and amino acid sequences are publicly available. Exemplary human STING nucleic acid sequences include GenBank Accession Nos. NM_198282, XM_011537640, XM_005268445, NM_001301738, and XM_011537639 and exemplary human STING amino acid sequences included GenBank Accession Nos. NP_938023, XP_011535942, XP_005268502, NP_001288667, and XP_011535941, all of which are incorporated herein by reference as of Feb. 23, 2016. Exemplary mouse STING nucleic acid sequences include GenBank Accession Nos. NM_001289591, NM_001289592, and NM_028261 and exemplary mouse STING amino acid sequences included GenBank Accession Nos. NP_001276520, NP_001276521, and NP_082537, all of which are incorporated herein by reference as of Feb. 23, 2016.

Molecules that bind to STING are referred to herein as “STING ligands” and molecules that bind to and activate STING are referred to as “STING agonists.” STING agonists include cyclic dinucleotides and derivatives thereof, such as modified cyclic dinucleotides. STING agonists also include xanthenone and derivatives thereof, including flavone acetic acid (FAA), xanthene-acetic acid (XAA), dimethylxanthenone-4-acetic acid (DMXAA), and derivatives thereof. As used herein, “STING pathway agonists” refers to molecules that activate STING by binding molecules upstream in the STING-dependent signaling pathway.

Subject: Living multi-cellular vertebrate organisms, a category that includes both human and non-human mammals. In some examples, a subject has a pre-malignant or virally-induced growth.

Treating or inhibiting: “Treating” a condition refers to a therapeutic intervention that ameliorates a sign or symptom of a disease or pathological condition, for example, a pre-malignant or virally-induced growth, after it has begun to develop. As used herein, the term “ameliorating,” refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. “Inhibiting” refers to inhibiting the full development of the disease or condition. Inhibition of a condition can span the spectrum from partial inhibition to substantially complete inhibition (e.g., including, but not limited to prevention) of the condition. In some examples, the term “inhibiting” refers to reducing or delaying the onset or progression of a disease. A subject to be administered a therapeutically effective amount of the disclosed compositions can be identified by standard diagnosing techniques for such a disorder, for example, based on signs and symptoms, family history, or risk factor to develop the disease or disorder.

Virally-induced growth: A mass or tissue growth caused by a viral infection. Some virally-induced growths are benign, while others may be pre-malignant. Exemplary virally-induced growths include those caused by infection with human papilloma virus (HPV), such as papillomas, condylomas, warts, or veruccas. As used herein, a virally-induced growth also include sores or papules caused by herpes virus (e.g., oral or genital sores), varicella virus (e.g., chickenpox or shingles lesions), and molluscum contagiosum virus. In additional examples, a virally-induced growth includes Merkel cell carcinoma.

### II. Methods of Treating or Inhibiting Virally-Induced or Pre-Malignant Growths or Benign Neoplasias

Disclosed herein are methods of treating or inhibiting virally-induced growths, pre-malignant conditions or growths, and/or benign neoplasias. In some examples, the pre-malignant growths or virally-induced growths include basal cells or develop from basal cells. In some embodiments, the methods include administering an effective amount of one or more STING agonists or a pharmaceutically acceptable salt or prodrug thereof to a subject who has a virally-induced growth, a pre-malignant growth, or a benign neoplasia.

A. STING Agonists

The methods disclosed herein utilize STING agonists. STING (Stimulator of Interferon Genes) is a cytosolic sensor of microbial infection. In particular, STING is an innate immune sensor of cyclic dinucleotides, which are believed to be produced only by bacteria and archaea (Burdette et al., Nature 478:515-518, 2011). Binding of CDNs to STING induces interferon (IFN) expression and inflammatory responses.

In some examples, STING agonists include compounds that bind to STING and increase expression and/or secretion of interferon (such as IFN-β), for example in macrophage cells. In other examples, STING agonists include compounds that bind to STING and activate the TBK1-IRF3 pathway, for example increasing TBK1 and/or IRF3 phosphorylation or activity. In each case, an increase in expression, secretion, or phosphorylation can be determined by comparison to a control, such as an unstimulated cell, or a cell contacted with a known STING agonist or a compound known not to bind to or activate STING. Binding of compounds to STING can be detected by determining binding of a radiolabeled compound to STING, for example utilizing UV crosslinking or competitive binding assays. Methods of detecting expression or secretion of IFN include reporter gene assays, RT-PCR assays, and antibody-based assays (such as ELISA). Protein phosphorylation assays, for example, using Western blot-based assays can be used to detect TBK1 or IRF3 phosphorylation.

In some examples, STING agonists are cyclic purine dinucleotides (such as CDNs produced by bacteria). Exemplary cyclic purine dinucleotides include cyclic diadenylate monophosphate (CDA), cyclic diguanylate monophosphate (CDG), cyclic di-inosine monophosphate (CDI), cyclic guanosine monophosphate-adenosine monophosphate (CGAMP), cyclic adenosine monophosphate-inosine monophosphate, and cyclic guanosine monophosphate-inosine monophosphate. In some examples, the disclosed CDNs are substantially pure stereoisomers (e.g., Rp,Rp or Rp,Sp stereoisomers of a CDN). Stereoisomers are molecules that have the same molecular formula and sequence of bonded atoms and which differ only in the three-dimensional orientation of the atoms in space.

STING agonists also include modified CDNs or derivatives of CDNs. The modified or derivative CDNs retain at least one activity of CDN STING agonists, such as binding to STING and increasing expression and/or secretion of interferon (such as IFN-β) and/or activating the TBK1-IRF3 pathway. In some examples, modified CDN or CDN derivative STING agonists include CDN thiophosphate compounds. One exemplary CDN thiophosphate compound is dithio-CDG (RR or RS stereoisomers). In another example, a modified STING agonist includes fluorinated CDNs, such as CDNs with a fluorine atom at the 2′ position of the nucleoside (e.g., c-di[2′FdGMP]). Additional exemplary modified CDNs or CDN derivatives that are STING agonists include those disclosed in U.S. Pat. App. Publ. No. 2014/0205653, incorporated herein by reference in its entirety.

Additional STING agonists include murine-specific STING agonists such as 5,6-dimethylxanthenone-4-acetic acid (DMXAA), flavone acetic acid (FAA) and 10-carboxymethol-9-acridanone (CMA; Cavlar et al., EMBO J. 32:1440-1450, 2013). Derivatives of these compounds that bind to and activate human STING are contemplated as being useful in the methods disclosed herein.

In additional examples, STING pathway agonists, which act by binding and/or activating molecules that are upstream of STING in the signaling cascade (e.g., molecules that activate STING and/or form a complex with STING, activating the downstream pathway), may also be used in the disclosed compositions and/or methods. Exemplary STING pathway agonists include molecules that bind to and/or activate cyclic GMP-AMP synthase (cGAS), DEAD-box helicase 41 (DDX41), DNA-dependent activator of IRFs (DAI), or gamma interferon-inducible protein 16 (IFI16). Agents targeting these molecules may result in STING-dependent activity similar to STING agonists that directly bind to and activate STING.

B. Methods of Treatment

In some embodiments, the disclosed methods include administering one or more STING agonists or a pharmaceutically acceptable salt or prodrug thereof to a subject with a pre-malignant growth or condition, a benign neoplasia, or a virally-induced growth. In some examples, pre-malignant growths or conditions include a mass or tissue that is potentially, but not yet, malignant. In some embodiments, the methods also include selecting a subject with a pre-malignant condition, a benign neoplasia, or a virally-induced growth who is to be treated with the one or more STING agonists.

Benign neoplasias are a mass of cells that do not have the ability to invade neighboring tissue or metastasize, and are therefore considered to be non-cancerous. One non-limiting example of a benign neoplasia is a keloid. Keloids are scars that form at the site of an injury and spread beyond the borders of the original injury. They include collagen and fibroblasts.

Examples of pre-malignant growths or conditions include intraepithelial neoplasia (e.g., intraepithelial neoplasia of the cervix (also referred to as cervical dysplasia), anus, vagina, penis, or vulva), pancreatic intraepithelial neoplasia, indraductal papillary mucinous neoplasm, pre-malignant growths of the breast (e.g., ductal carcinoma in situ, atypical ductal hyperplasia, atypical lobular hyperplasia), extramammary Paget disease (non-invasive intraepithelial adenocarcinoma), actinic keratosis, colon polyps, leukoplakia, erythroplakia, lichen planus (e.g., oral or genital), atypical nevus, lung adenocarcinoma in situ (also referred to as bronchioloalveolar carcinoma), and Barrett's esophagus.

Virally-induced growths include benign or pre-malignant growths caused by viral infection, such as infection with one or more subtypes of human papillomavirus (HPV). Examples of virally-induced growths include papillomas, condylomas, verrucas, or warts (such as common warts and plantar warts). Virally-induced growths also include sores or papules caused by herpes virus (e.g., oral or genital sores), varicella virus (e.g., chickenpox or shingles lesions), and molluscum contagiosum virus.

STING agonists (for example, a CDN or derivative or analog thereof) or a pharmaceutically acceptable salt or prodrug thereof can be administered to a subject in need of treatment using any suitable means known in the art. Methods of administration include, but are not limited to, intradermal, transdermal, intramuscular, intraperitoneal, parenteral, intravenous, subcutaneous, intratumoral, vaginal, rectal, intranasal, inhalation, or oral administration. In some examples, the STING agonist is administered by direct injection (for example, injection at or near the site of a pre-malignant or virally induced growth) or locoregional administration (for example via a gel or controlled release formulation).

Parenteral administration (including direct injection) is generally achieved by injection. Injectables can be prepared in conventional forms, either as liquid solutions or suspensions, solid forms suitable for solution of suspension in liquid prior to injection, or as emulsions. Injection solutions and suspensions can be prepared from sterile powders, granules, and tablets. Administration can be systemic or local (for example, injection or topical application at or near the site of a pre-malignant growth or a virally-induced growth).

The one or more STING agonists are administered in any suitable manner, preferably with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure. The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, Pa., 21^{st} Edition (2005) describes compositions and formulations suitable for pharmaceutical delivery of one or more therapeutic agents

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media (such as phosphate buffered saline). Parenteral vehicles include but are not limited to sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Compositions for oral administration include powders or granules, suspensions or solutions in water or non-aqueous media, capsules, sachets, or tablets. Thickeners, flavorings, diluents, emulsifiers, dispersing aids or binders may be desirable. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

In some embodiments, liposomes are used to deliver one or more STING agonists to a subject. Suitable liposomes for use in the compositions and methods disclosed herein can be formed from standard vesicle-forming lipids, which generally include neutral or negatively charged phospholipids and a sterol, such as cholesterol. The selection of lipids is generally guided by consideration of several factors, such as the desired liposome size and half-life of the liposomes.

Appropriate dosages for treatment with one or more STING agonists can be determined by one of skill in the art. In general, an effective amount of a STING agonist administered to a subject will vary depending upon a number of factors associated with that subject, for example the overall health of the subject, the condition to be treated, or the severity of the condition. An effective amount of a STING agonist can be determined by varying the dosage of the compound and measuring the resulting therapeutic response, such as a decrease in the size, volume, or number of pre-malignant, benign, or virally-induced growth(s). Alternatively, response can be assessed by measuring cytokine or other markers of inflammation released locally, or measured systemically in the blood. In other examples, response is assessed by visual inspection of the growth, for example, changes in swelling, redness, temperature changes, and/or blackening of all or portions of the growth.

In particular examples, the one or more STING agonists or derivative or prodrug thereof is administered intravenously, intraperitoneally, subcutaneously, or orally. In other examples, the one or more STING agonists or derivative or prodrug thereof is administered at or near the site of the pre-malignant growth or virally-induced growth, for example by local injection or topical application. Dosages for administration to a human subject can be determined by a person of skill in the art, for example, based on dose translation from mouse studies, and subsequent testing in clinical trials.

In some non-limiting examples, the dose of a STING agonist administered to a subject is about 0.1 mg/kg to about 1000 mg/kg. In particular examples, the dose may be about 0.1 mg/kg to about 100 mg/kg, such as about 0.5 mg/kg, 1 mg/kg, 5 mg/kg, 10 mg/kg, 20 mg/kg, 40 mg/kg, 50 mg/kg, 75 mg/kg, or 100 mg/kg. In other examples, the dose may be about 10 to 1000 mg, for example, about 20 mg to 500 mg, or about 50 mg to 800 mg of a STING agonist. In still further examples, the dose may be about 1 μg to 10 mg, for example, about 10-50 μg, about 25-100 μg, about 50-250 μg, about 100-500 μg, about 250-1000 μg, about 500 μg to 2.5 μmg, or about 1-10 mg. In additional examples, the dose may be about 25 μg, about 50 μg, about 75 μg, about 100 μg, about 200 μg, about 250 μg, about 300 μg, about 400 μg, about 500 μg, about 600 μg, about 700 μg, about 750 μg, about 800 μg, about 900 μg, about 1 mg, or more.

In other non-limiting examples, a STING agonist is administered to a subject by direct injection at or near a lesion at about 0.1 μg/mm² to about 100 μg/mm² (e.g., about 0.1 μg/mm² to about 1 μg/mm², about 0.5 μg/mm² to about 5 μg/mm², about 2.5 μg/mm² to about 10 μg/mm², about 5 μg/mm² to about 50 μg/mm², about 25 μg/mm² to about 80 μg/mm², or about 75 μg/mm² to about 100 μg/mm²), based on the size of the growth or lesion. These doses may administered through distributed injections, where the lesion may be subdivided into zones for multiple injections, for example each 5 mm² zone may receive a single injection so that a 25 mm² lesion may receive 5 separate injections. In this case there will be a dose per injection and an overall dose scaled by lesion size.

The one or more STING agonists can be administered in a single dose, or in several doses, as needed to obtain the desired response. However, the effective amount can be dependent on the specific STING agonist(s), the subject being treated, the severity and type of the condition being treated, and the manner of administration. In some examples, a dose of a STING agonist is administered daily, weekly, bi-weekly, or monthly, once or more (such as 1, 2, 3, 4, 5, or more doses). In some examples, the effectiveness of the one or more STING agonists is monitored by observing the pre-malignant or virally-induced growth over time (for example, size, volume, and/or number of growths). Subjects who are found to have been poorly or partially responsive to the initial administration are given one or more additional doses of the one or more STING agonists. Thus, in some examples, weekly cycles of administration may result in loss of disease and will reveal regions of disease that did not initially respond or did not receive injection (e.g., in the case of bulky disease). In this case repeated cycles of treatment may be applied until all disease is lost or until residual material can suitably be removed through techniques such as surgical resection or loop excision.

The disclosed methods include STING agonists, which can be administered alone, in the presence of a pharmaceutically acceptable carrier, in the presence of other therapeutic agents or interventions, or both. A clinician of ordinary skill in the art can identify appropriate additional treatments for a subject based on the type of condition being treated. In non-limiting examples, a subject with DCIS may also be treated with surgical excision, endocrine therapy, and/or radiation therapy, while a subject with plantar warts may also be treated with liquid nitrogen with skin shaving.

The following examples are provided to illustrate certain particular features and/or embodiments. These examples should not be construed to limit the disclosure to the particular features or embodiments described.

### EXAMPLE 1

### Treatment of Papilloma with STING Agonist in Mice

This example describes treatment of murine papilloma with a cyclic dinucleotide STING agonist.

### Methods

Pdx-Cre^{+/-} (Stock190 014647, Jackson Laboratories, Bar Harbor, Me.), Kras^{(G12D)+/-} (Stock#008179, Jackson Laboratories), and Trp53^{(R172H)+/-} (Stock#01XM2, NCI Fredrick Mouse Repository) mice were crossed to generate Pdx-Cre^{+/-} Kras ^{(G12D)+/-} Trp53^{(R172H)+/-} that generate pancreatic tumors (Hingorani et al., Cancer Cell 7:469-483, 2005). At variable penetrance, mice develop papilloma of the face and anogenital region (Hingorani et al., Cancer Cell 7:469-483, 2005; Gades et al., Comp. Med. 58:271-275, 2008). Mice bearing papilloma with no evidence of pancreatic tumors and less than 90 days of age were accrued to this study. Masses were treated by injection of 25 μg of c-di-CDG (Invivogen, San Diego, Calif., Catalog No. tlrl-naldg), Imiquimod (Invivogen) or PBS vehicle.

### Results

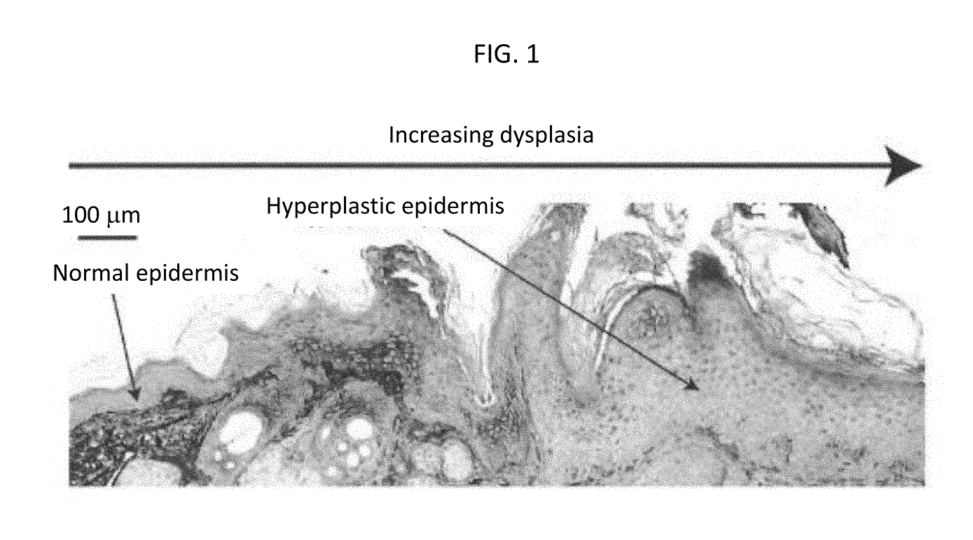

These experiments utilized a transgenic mouse model carrying the mutant tumor-driving genes Kras^{(G12D)} and p53^{(R172H)} that are controlled by expression of PDX-Cre. This results in progressive carcinogenesis through pancreatic intraepithelial neoplasia to pancreatic ductal adenocarcinoma (Hingorani et al., Cancer Cell 7:469-483, 2005). In addition, these mice spontaneously develop papilloma of the face and vulva, as shown in FIG. 1, where in places the normal skin undergoes epidermal thickening that closely resembles HPV-associated papilloma in humans.

The Pdx-Cre^{+/-} Kras^{(G12D)+/-} Trp53 ^{(R172H)+/-} mice were observed to develop papilloma at variable penetrance, as has been previously reported in the literature (Hingorani et al., Cancer Cell 7:469-483, 2005; Gades et al., Comp. Med. 58:271-275, 2008). We found no association between papilloma formation and progression of pancreatic adenocarcinoma in the mice, and generally the papilloma was present before progression to invasive carcinoma in the pancreas. Some mice developed more than one papilloma, with the location restricted to the periauricular and anogenital regions. As previously reported, papilloma formation required both PDX-Cre and Kras^{(G12D)} genotypes, suggesting a genetic origin (Gades et al., Comp. Med. 58:271-275, 2008). Prior studies failed to find an infectious origin for these papilloma (Gades et al., Comp. Med. 58:271-275, 2008), and in our colony papilloma were never found in PDX-Cre⁻ animals nor PDX-Cre⁺ Trp53^{(R172H)+/-} mice that lacked Kras^{(G12D)}, despite co-housing. Mice were unperturbed by the papilloma, and though individual papilloma could be large, we found no evidence of progression to invasive carcinoma at the site. However, progression of pancreatic adenocarcinoma in these mice precluded long-term follow up of the papilloma. Histological analysis of the papilloma demonstrated significant thickening of the skin with formation of classical keratinized papilloma as reported (Gades et al., Comp. Med. 58:271-275, 2008).

STING ligands were administered into the papilloma by direct injection with a total dose of 25 μg administered in a 25 μl injection volume. In the control case, 25 μg of Imiquimod was administered using the same technique. For larger papillomas, the injection was split between different sites within the same growth. The total dose to the growth remained 25 μg in each case. The animals were retreated on day 7 with a second cycle of injections.

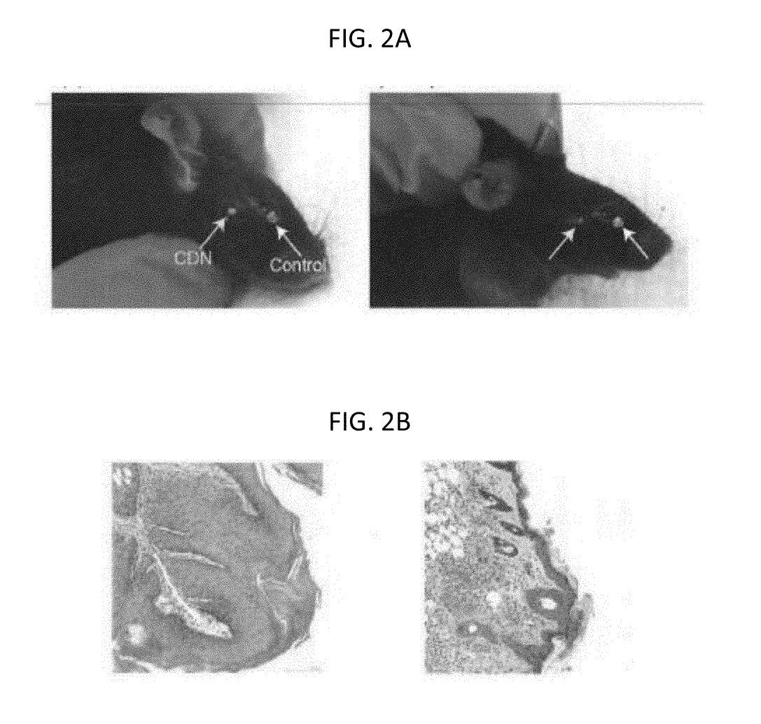

Rapid alterations in small papilloma to resemble normal skin were observed within 18 hours of treatment (FIGS. 2A and B). In pilot studies, mice bearing papilloma were randomly assigned to treatment with the STING ligand c-di-GMP (CDG) or PBS vehicle control. In an example of a mouse with two small papilloma on the face, one was treated by direct injection of CDG, and the other given vehicle control (FIG. 2A, left). The CDG-injected papilloma rapidly reverted to normal skin within 18 hours (FIG. 2A, right). The site exhibited slight reddening, and histological analysis demonstrated an inflammatory infiltrate in the subcutaneous space (FIG. 2B). Injection of STING ligand into histologically normal skin at distant sites or in normal mice had no observable effect (not shown). Thus, CDG was non-toxic at these doses and caused rapid, site-specific regression of experimental papilloma.

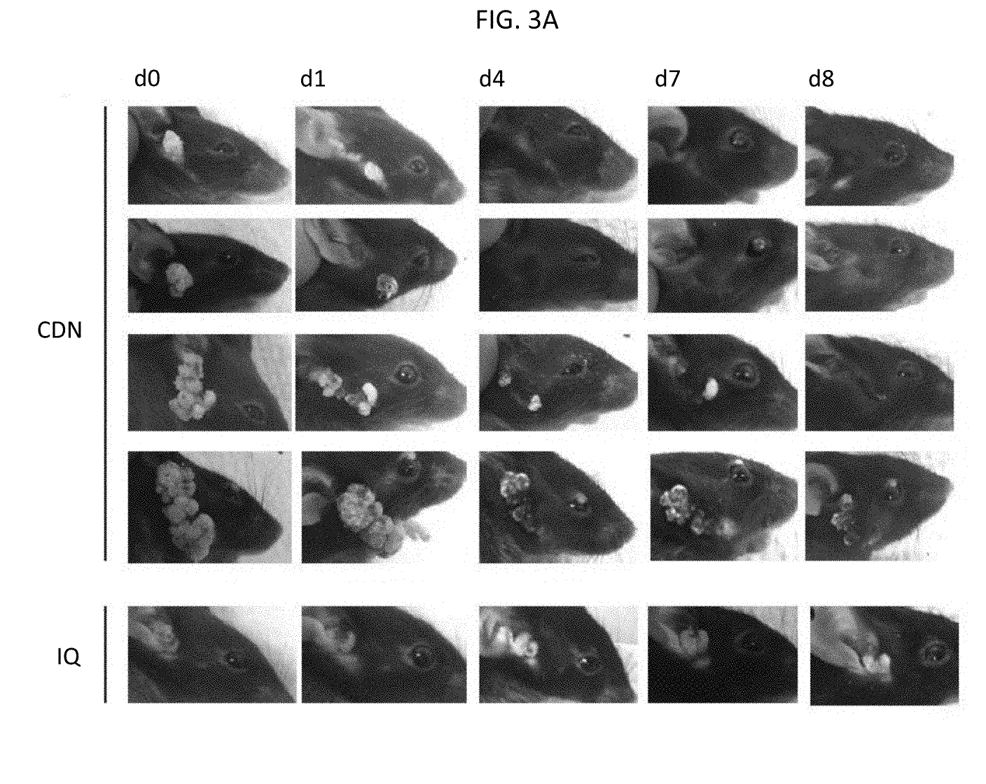

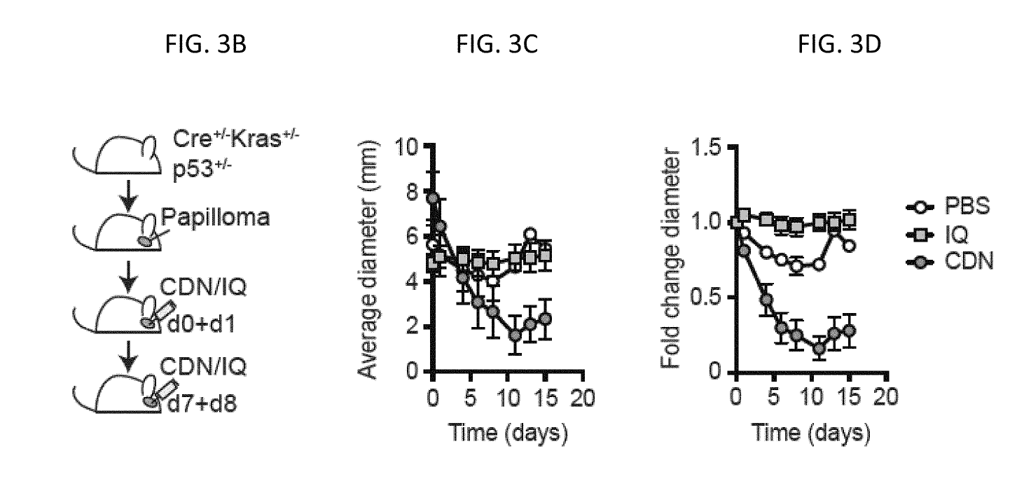

In addition, very large papilloma was treated with two cycles of treatment separated by 1 week, resulting in dramatic tumor regression (FIG. 3A). Importantly, this effect was dramatically more effective than the existing Imiquimod treatment (FIG. 3A). We tested treatment of larger, 3-15 mm papilloma with STING ligands. A single injection of CDG resulted in rapid loss of papilloma around the injection site, but these did not fully regress. Therefore, we developed a treatment course consisting of injections on d0 and d1, and again on d7 and d8 as the papilloma decreased in size (FIG. 3B). Mice were randomized to receive CDG, to conventional treatment with the TLR7 ligand imiquimod, or PBS vehicle. While imiquimod did not significantly affect the size of the papilloma, CDG treatment resulted in significantly decreased papilloma size (FIGS. 3B and C). The CDG-treated papilloma showed evidence of blackening (FIG. 3A), which was suggestive of the hemorrhagic necrosis previously observed in STING ligand treatment of advanced cancers (Baird et al., Cancer Res. 76:50-61, 2016). Treatment resulted in complete regression of some papilloma, with skin returning to normal appearance without skin breaks and with the return of hair.

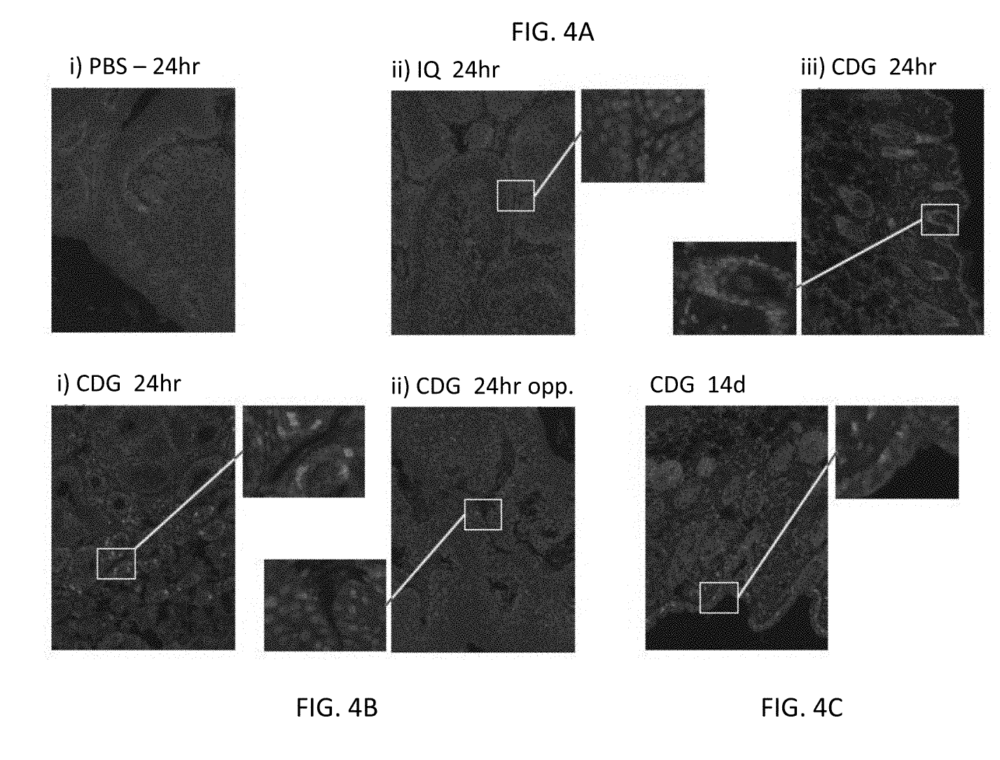

Histological examination of CDG-treated papilloma demonstrated regions of inflammatory infiltrate, but no major areas of necrosis (not shown). To investigate the effect of treatment on T cell infiltration, which we and others have shown play a role in clearance of advanced cancers following administration of STING ligands (Baird et al., Cancer Res. 76:50-61, 2016; Corrales et al., Cell Rep. 11:1018-1030, 2015), papilloma were stained for infiltrating CD3⁺ T cells. Few to no T cells were detected in PBS or imiquimod-treated papilloma (FIG. 4A, i-ii), but 24 hours following CDG treatment CD3⁺ T cells were found associated with the normalized skin epithelium (FIG. 4A, iii). In examples where mice exhibited more than one papilloma, CDG treatment of one papilloma resulted in T cell infiltration and control only in the treated papilloma (FIG. 4B). T cells remained associated with papilloma for extended periods, and were enriched in the normalized skin 14 days following treatment (FIG. 4C).

### EXAMPLE 2

### Effect of STING Agonists on Macrophages

This example describes the effect of STING agonists on murine bone marrow-derived macrophages.

Murine bone marrow-derived macrophages were treated for 24 hours with a range of immunological adjuvants targeting STING (CDA), NOD2 (PGNECndss), TLR7 (imiquimod), TLR9 (class A CpG), or RIGI (ppp5′dsRNA), then treated with IFNγ (20ng/ml) or IL-4 (20ng/ml) to direct M1 or M2 differentiation, respectively. Ligation of TLR4 (LPS) was a positive control. M1 differentiation was distinguished by expression of iNOS, and M2 differentiation by expression of arginase I as measured by Western blotting.

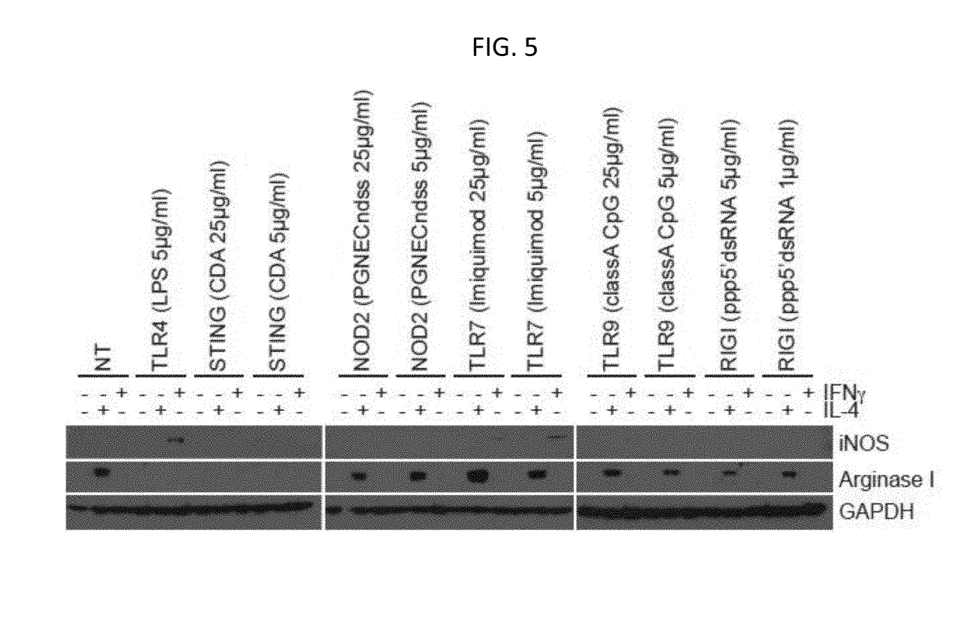

Administration of CDA to bone marrow-derived macrophages prevented M2 differentiation by IL-4 without driving M1 differentiation (FIG. 5). This is distinct from imiquimod, which can drive macrophages into either M1 or M2 differentiation. Without being bound by theory, it is generally thought that M2 differentiated macrophages support invasive tumors.

### EXAMPLE 3

### STING Expression in Normal Tonsil and Benign and Premalignant Oral Dysplasia

### Methods

Archived tissue blocks were obtained and 5μm sections were cut and mounted for analysis. Tissue sections were boiled in EDTA buffer for antigen retrieval. Sections were first stained with rabbit anti-STING (Cell Signaling Technologies, Danvers, Mass.) and primary antibody binding was detected with HRP conjugated secondary antibodies followed by DAB development and counterstaining. Images were acquired using a Leica SCN400 whole slide scanner.

Representative blocks of archived tissue from approximately five patients with each histology were obtained and sectioned. To represent distinct types of oral dysplasia, we identified examples of benign dysplasia, inflammatory conditions including lichen planus and candida ulcer, as well as examples of mild, moderate and sever dysplasia. To represent HPV-associated dysplasia and pre-malignancy we identified examples of benign dysplasia, condyloma, anal intraepithelial neoplasia grade 3 (AIN3), and cervical intraepithelial neoplasia grade 3 (CIN3). Tissues were sectioned and stained for STING as above. The degree of STING staining was scored by pathologist.

### Results

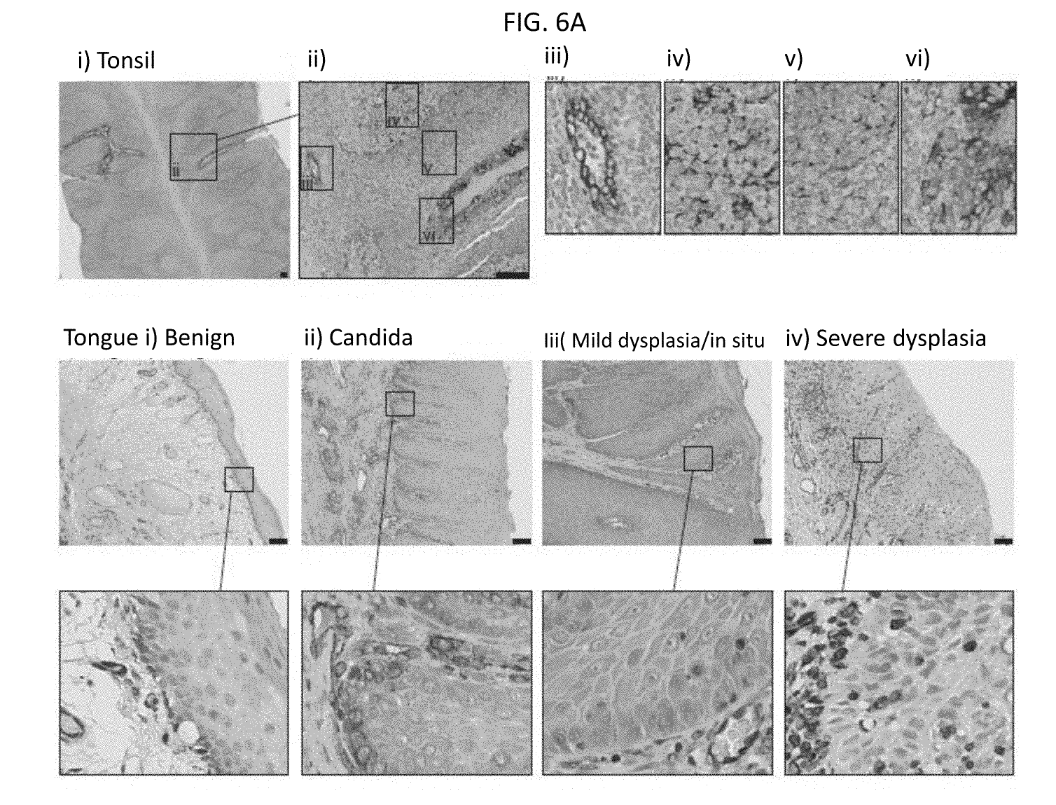

We examined STING expression in normal human tonsil and in a panel of human benign and premalignant oral dysplasia. Representative examples are shown in FIGS. 6A and B. Immunohistology demonstrated strong expression of STING in cells in the tonsil (FIG. 6A, i-ii), in particular strongly staining high endothelial venules (FIG. 6A, iii), cells within the light zone of the germinal center that are consistent with follicular dendritic cells (FIG. 6A, iv), and cells within the T cell zone that are consistent with inderdigitating cells (FIG. 6A, v). STING expression was readily detectible in immune cells underlying normal tonsillar epithelia, but was particularly high in select tonsillar crypts (FIG. 6A, i and vi), suggestive of ongoing responses to bacterial or viral infection in specific sites resulting in increased STING expression in squamous cells.

In normal tongue, STING expression was restricted to the basal cell layer and lost on differentiation into keratinocyte layers (FIG. 6B, i). In areas of oral candidiasis associated with benign thickening, STING expression was occasionally enhanced (FIG. 6B, ii). In mild or severe dysplasia of the tongue, STING expression was mixed but did not increase with the degree of dysplasia (FIG. 6B, iii-iv), and occasionally STING expression was not detected in the squamous cells of severe dysplasia (FIG. 6B, iv). However, in each case, expression of STING was readily detectable in endothelial cells underlying the squamous layer and in immune cells underlying the squamous layer. These data show that STING is expressed by basal cells in the oral mucosa, which are a key target for HPV infection, but there is no significant association with STING expression and the progression of dysplasia in the tongue or oral cavity.

### EXAMPLE 4

STING Expression in HPV-Associated Pre-malignancies and Cancers Methods

A panel of paraffin embedded tissue blocks was used to generate an array of tumors of mixed HNSCC origins using a Perkin Elmer tissue microarrayer. Patient tumors were classified as HPV⁺ if they scored as positive for p16 and originated in the oropharynx. Samples that were negative for p16 and/or originated outside the oropharynx were scored as HPV⁻. Arrayed tissues were sectioned and stained for STING as above. The STING staining score was determined by automated image analysis of tumors, grading the number of STING positive cancer cells and their staining intensity to generate an expression score.

### Results

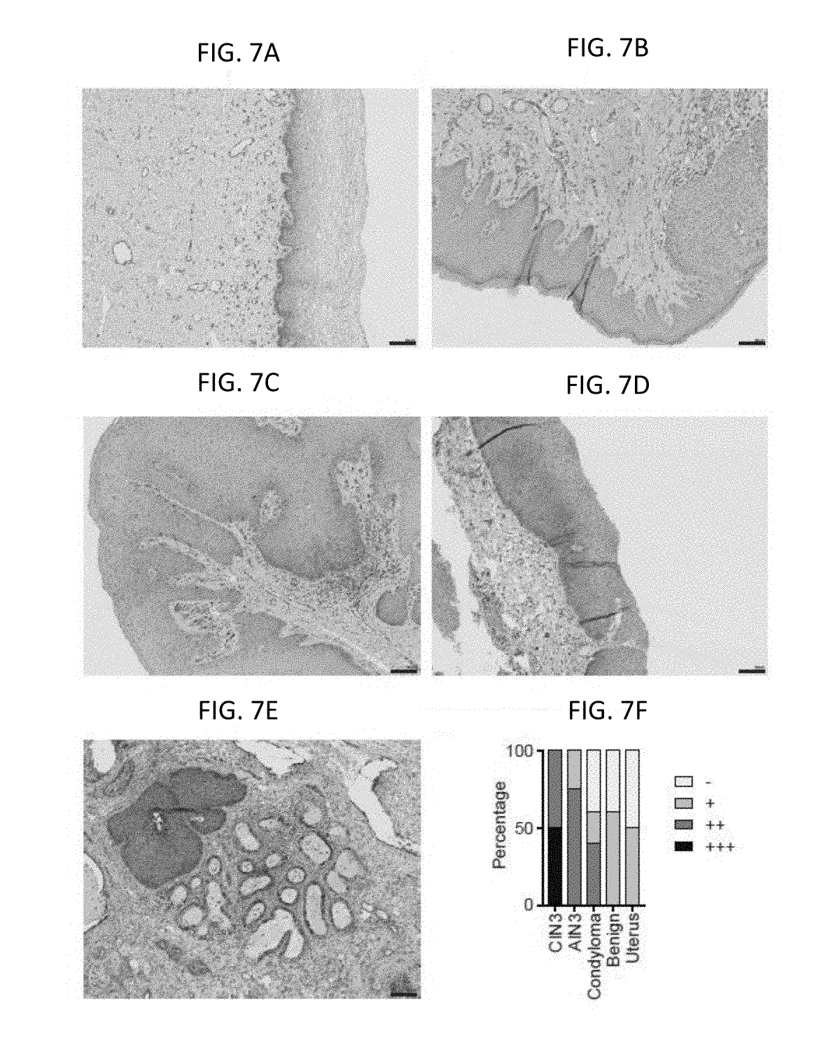

While HPV is known to result in HPV-associated cancers in the tonsil, pre-malignant HPV⁺ papilloma are rarely discovered in the tonsil. To evaluate STING expression in HPV-associated pre-malignancies, we performed immunohistology of high grade cervical intraepithelial neoplasia (CIN3), high grade anal intraepithelial neoplasia (AIN3), and condyloma, as well as benign hyperplasia and tissues from normal uterus as a control. The pattern of normal tissue STING expression was very similar to that seen in the tonsil and tongue, with predominant expression in the basal cells and underlying immune cells, and loss on differentiation into the keratinocyte layer (FIGS. 7A-7E). STING was also expressed in all HPV-associated dysplasia, with a trend towards increased expression of STING in CIN3 (FIG. 7F). In addition, in all cases, STING was expressed in endothelial structures and immune cells underlying the squamous tissue. These data demonstrate that STING is expressed in the key target cells of HPV and expression is maintained or increased in these cells through HPV⁺ dysplastic progression.

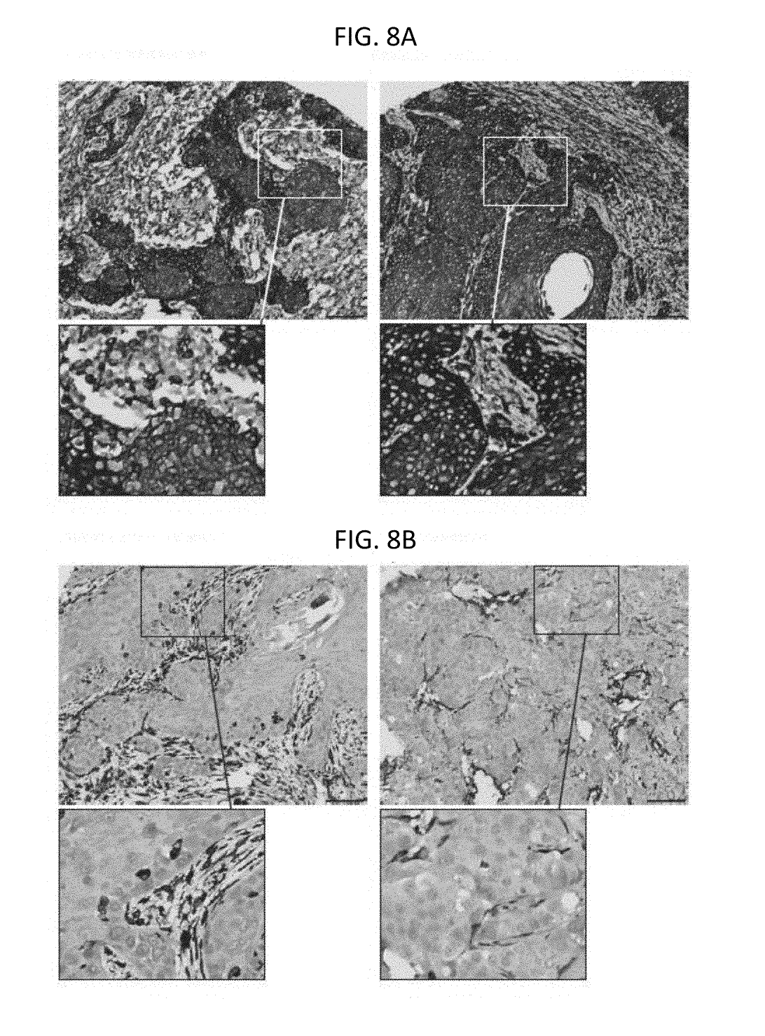

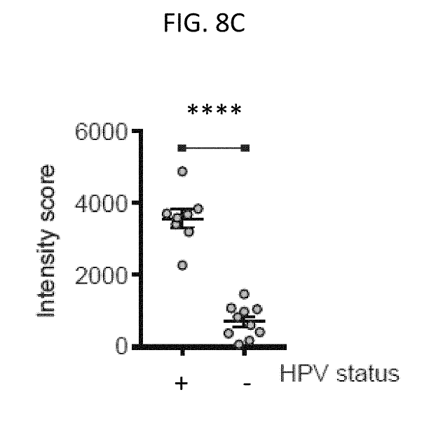

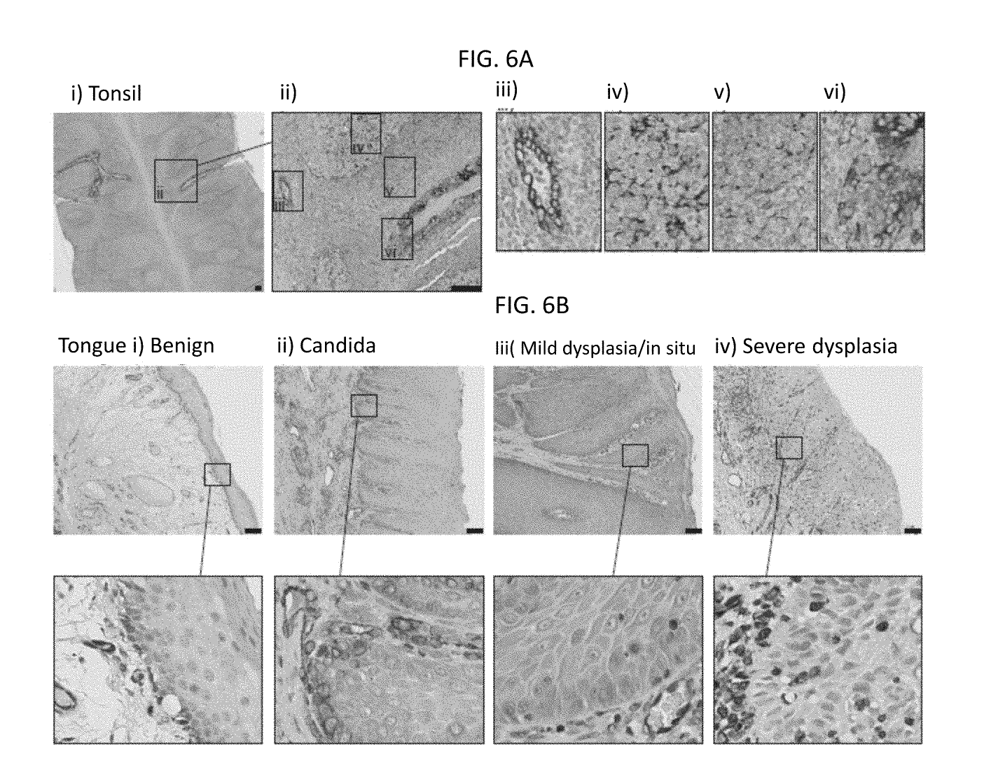

To examine STING expression in advanced HPV⁺ and HPV⁻ cancers, we stained a tissue array from a panel of mixed HNSCC origins. The tumors were classified as HPV⁺ if they scored as positive for p16 and originated in the oropharynx. Samples that were negative for p16 and/or originated outside the oropharynx were scored as HPV⁻. In all cases, regardless of cancer cell expression, STING was detectable in immune cells in the vicinity of the tumor. However, HPV⁺ HNSCC exhibited high levels of STING expression in the cancer cells (FIG. 8A), while HPV⁻ HNSCC exhibited low or absent STING expression (FIG. 8B). These samples were assigned an expression score according to the proportion of cancer cells with high STING staining in the cytoplasm, using Definiens image analysis software (Definiens, Cambridge, Mass.). These data show that HPV⁺ HNSCC exhibited significantly higher STING expression in cancer cells than HPV⁻ HNSCC (FIG. 8C). In view of the basal origin of HPV⁺ cancer cells, these data suggest that HPV⁺ cancer cells preserve their STING expression through malignant progression from their basal cell origin. This contrasts to the non-basaloid squamous HPV⁻ HNSCC that poorly express STING, consistent with the loss of STING expression in the more differentiated cells of the normal squamous epithelium. These data demonstrate that STING expression is a strong marker of HPV⁺ HNSCC.

### EXAMPLE 5

### Treatment of Human Papillomavirus-Associated Disease in an Animal Model

This example describes representative methods for treating HPV-associated pre-malignant disease or HPV-associated squamous cell carcinoma in an animal model. However, one skilled in the art will appreciate that methods that deviate from these specific methods can also be used to successfully treat or inhibit HPV-associated disease in an animal model.

Mice transgenic for HPV16 E6 protein and/or E7 protein are a model for progressive neoplastic diseases, including invasive squamous cell cervical carcinoma, head and neck squamous cell carcinoma (HNSCC), and squamous cell carcinoma of the anus, depending on the tumorigenic trigger utilized (Riley et al., Cancer Res. 63:4862-4871, 2003; Strati et al., Proc. Natl. Acad. Sci. USA 103:14153-14157, 2006).

Mice that develop pre-malignant disease through these transgenic drivers are accrued to the study based on development of a measurable, accessible pre-malignant lesion. Lesions are treated with multiple cycles of direct injection of STING ligand, for example CDG, at an initial dose of 25 μg in 25 μl vehicle. Additional dosages are tested (in the same or different mice) based on the efficacy of the initial dose. Control mice or control lesions are treated with vehicle alone or Imiquimod as a comparison. The dose of STING ligand may be distributed across multiple injections in larger lesions. One cycle consists of a single time point of injection, with 7 days of follow-up. Those with detectable residual disease are administered further cycles of treatment. In some examples, mice are randomized to receive surgical excision of residual disease following STING ligand administration. In each case, the outcome is monitored by immunohistological assessment of treated and control lesions, multiplex assay of local and systemic cytokine and chemokine release, and long term follow-up to measure local recurrence and rate of transformation to malignant disease.

### EXAMPLE 6

### Methods of Treating or Inhibiting Pre-Malignant Growths

This example provides exemplary methods for treating or inhibiting a pre-malignant growth in a subject. However, one skilled in the art will appreciate that methods that deviate from these specific methods can also be used to successfully treat or inhibit pre-malignant growths in a subject.

In particular examples, the method includes selecting a subject with a pre-malignant growth, such as a pre-malignant growth of the cervix. In the case of pre-malignant growth in the cervix, patients are accrued by physicians following a positive PAP smear or similar screening procedure. Those eligible for the study are randomized to administration of a STING agonist, vehicle, or a control agent such as imiquimod, and this would be followed by surgical removal, cryoablation, or loop excision, according to physician preference.

Subjects selected for treatment are administered a STING agonist, such as a CDN molecule (for example, CDG or CDA). In some examples, a CDN is administered to the subject at doses of about 1 μg to 100 μg per direct injection. The STING agonist is administered in one or several doses, for example at weekly intervals. The mode of administration can be any used in the art, including but not limited to direct injection or administration of a gel or sustained release agent to the target site. The amount of agent administered to the subject can be determined by a clinician, and may depend on the particular subject treated. Specific exemplary amounts are provided herein (but the disclosure is not limited to such doses).

The size of the pre-malignant growth is monitored at time points following administration of the STING agonist. A decrease or lack of change in the size of the growth or decrease in number of growths is an indicator of efficacy of treatment. A lack of recurrence and/or lack of progression to malignancy are additional indicators of treatment efficacy. Where excision takes place, the disease is also examined by histology to compare immune infiltrates and tissue architecture in each treatment group.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A method of treating a subject with a pre-malignant growth or condition, a benign neoplasia, or a virally-induced growth, comprising administering to the subject with the pre-malignant growth or condition, benign neoplasia, or virally-induced growth a composition comprising an effective amount of one or more Stimulator of Interferon Genes (STING) agonists, or a pharmaceutically acceptable salt or prodrug thereof.

2. The method of claim 1, wherein the one or more STING agonists is a cyclic dinucleotide or a derivative thereof.

3. The method of claim 2, wherein the cyclic dinucleotide is cyclic diadenylate monophosphate (CDA), cyclic diguanylate monophosphate (CDG), cyclic di-inosine monophosphate (CDI), cyclic guanosine monophosphate-adenosine monophosphate (cGAMP), cyclic adenosine monophosphate-insoine monophosphate, or cyclic guanosine monophosphate-inosine monophosphate.

4. The method of claim 2, wherein the cyclic dinucleotide derivative is a cyclic dinucleotide thiophosphate. 20

5. The method of claim 4, wherein the cyclic dinucleotide thiophosphate is dithio-CDG or dithio-CDA.

6. The method of claim 2, wherein the composition comprises a substantially pure stereoisomer of the cyclic dinucleotide or derivative thereof.

7. The method of claim 1, wherein the subject has a pre-malignant condition comprising cervical dysplasia, intraepithelial neoplasia, ductal carcinoma in situ, actinic keratosis, colon polyps, leukoplakia, erythroplakia, lichen planus, or Barrett's esophagus.

8. The method of claim 1, wherein the subject has a virally-induced growth comprising a papilloma, a condyloma, a verruca, or a wart.

9. The method of claim 1, wherein the pre-malignant growth or condition or the virally-induced growth is caused by infection with a human papillomavirus, herpes virus, varicella virus, or molluscum contagiosum virus.

10. The method of claim 1, wherein the composition comprising the one or more STING agonists or a pharmaceutically acceptable salt or prodrug thereof is administered to the subject intravenously, orally, or subcutaneously.

11. The method of claim 1, wherein the composition comprising the one or more STING agonists or a pharmaceutically acceptable salt or prodrug thereof is administered to the subject at or near the site of the pre-malignant condition or growth or virally-induced growth.

12. The method of claim 11, wherein the composition comprising the one or more STING agonists or a pharmaceutically acceptable salt or prodrug thereof is administered by injection or topically.

13. The method of claim 1, further comprising selecting a subject with a pre-malignant condition or virally-induced growth for treatment.
